(19) United States
(12) Patent Application Publication
(10) Pub. No.: US 2012/0189547 A1
(43) Pub. Date: Jul. 26, 2012

(54) [18F] LABELLED ANALOGUES OF FLUMAZENIL AS IN VIVO IMAGING AGENTS

(76) Inventors: John Woodcraft, Amersham (GB); Clare L. Jones, Amersham (GB); Alessandra Gaeta, Amersham (GB); William John Trigg, Amersham (GB); Paul Alexander Jones, Amersham (GB); Stuart Plant, Amersham (GB)

(21) Appl. No.: 13/394,639

(22) PCT Filed: Oct. 8, 2010
(86) PCT No.: PCT/EP2010/065126

§ 371 Date: Apr. 10, 2012

Related U.S. Application Data
(60) Provisional application No. 61/250,890, filed on Oct. 13, 2009.

(30) Foreign Application Priority Data
Oct. 8, 2009 (GB) 0917612.4

(51) Int. Cl. A61K 51/04; B01J 19/00; C07D 487/02

(52) U.S. Cl. 424/1.89; 540/498; 422/129

(57) ABSTRACT

The present invention provides radiofluorinated compounds useful for in vivo imaging GABA_{A} receptors. Also provided by the present invention is a method of synthesis for the radiofluorinated compounds of the invention, in particular an automated method of synthesis. A further aspect of the invention is a cassette suitable for carrying out the automated method of synthesis of the invention.

### TECHNICAL FIELD OF THE INVENTION

The present invention relates to in vivo imaging and in particular to in vivo imaging of gamma-aminobutyric acid (GABA) receptors of the central nervous system (CNS). The invention provides novel radiofluorinated compounds based on the benzodiazepine antagonist flumazenil.

### DESCRIPTION OF RELATED ART

Gamma-aminobutyric acid (GABA) is the most important inhibitory neurotransmitter in the human brain. GABA receptors are transmembrane receptors and fall into two main types, GABA_{A} receptors and GABA_{B} receptors. GABA_{A} receptors have been the major focus of pharmalogical development to date. Many GABA_{A} receptor subtypes have been discovered and novel chemical structures have been developed which are selective for these subtypes. Normal activation of the GABA_{A} receptor results in chloride ion being selectively conducted through its pore. This chloride channel gating is generally inhibitory on a neuron by virtue of stabilising the membrane potential near to resting level.

Defective GABA_{A} receptor neurotransmission may be caused by a reduction in GABA_{A} receptors, or by defective functioning of the GABA_{A} receptor due to e.g. a genetic mutation in a GABA_{A} receptor gene, traumatic brain injury, or a pharmacological insult, and is implicated in a number of neurological and psychiatric disorders, including epilepsy, anxiety disorders, Parkinson's disease and chronic pain. The development of radioligands selective for the GABA_{A} receptor is therefore of value in terms of brain imaging studies in living human patients, in particular those suffering from disorders associated with defective GABA_{A} receptor neurotransmission.

Flumazenil (also known as flumazepil, code name Ro 15-1788, trade names Anexate, Lanexat, Mazicon, Romazicon) is an imidazo[1,5-a][1,4]benzodiazepine that is a neutralising allosteric modulator of GABA_{A} receptors in the CNS (Johnston 1996 Pharmacol Ther; 69(3): 173-198). The chemical structure of flumazenil is as follows:

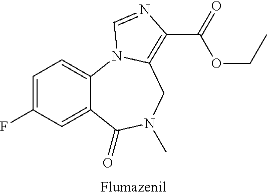

The most common use of flumazenil to date has been as an antidote to benzodiazepine overdose as it reverses the effects of benzodiazepines by competitive inhibition at the benzodiazepine binding site of the GABA_{A} receptor. In addition, because flumazenil has little or no agonist activity, radiolabelled versions thereof have been developed as positron emission tomography (PET) radiotracers.

Radiofluorinated derivatives of flumazenil known in the art are: [¹⁸F]flumazenil ([¹⁸F]FMZ); [¹⁸F]fluoroflumazenil ([¹⁸F]FFMZ); and, [¹⁸F]fluoroethylflumazenil ([¹⁸F]FEFMZ).

[¹⁸F]FMZ has the same chemical formula as flumazenil but wherein ¹⁸F is incorporated by direct radiofluorination of a nitro precursor compound:

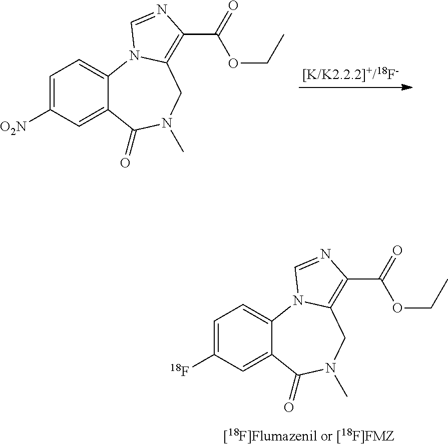

[¹⁸F]FMZ binds to the GABA_{A} receptor with high affinity (Kᵢ around 0.5 nM) and selectivity. Ryzhikov et at (2005 Nuc Med Biol; 32: 109-116) describe the preparation of [¹⁸F]FMZ from a nitro precursor compound. This synthesis, however, has been found by the present inventors to have a less than optimal end of synthesis (EOS) yield of 2.7-7.7% (described herein as a comparative example). Furthermore, the synthesis as described by Ryzhikov et at uses a high reaction temperature which is not amenable to automation on all radiosynthesis platforms. These EOS yields are comparable to those reported by Odano et at (Neuroimage 2009 45(3) 891-902).

[¹⁸F]FFMZ is an ¹⁸F-labelled derivative of flumazenil wherein ¹⁸F is incorporated by fluoroethylation of a carboxylic acid precursor compound (Mitterhauser et at 2004 Nuc Med Biol; 31: 291-295):

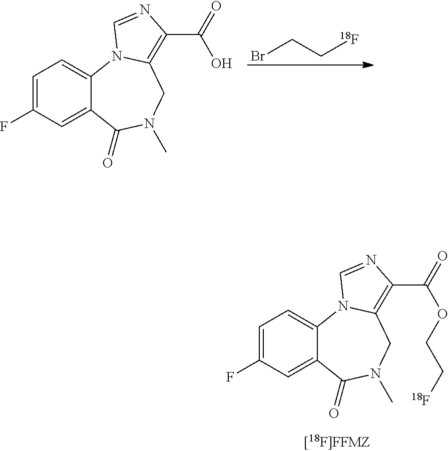

[¹⁸F]FFMZ is reported as having high brain uptake and high selective binding to GABA_{A} receptors. However, the synthesis of [¹⁸F]FFMZ results in a low EOS yield.

[¹⁸F]FEFMZ can be obtained by N-alkylation of a desmethyl precursor compound using [¹⁸F]fluoroethyltosylate in a one-pot synthesis (Moerlein and Perlmutter 1992 Eur J Pharmacol; 218: 109-115):

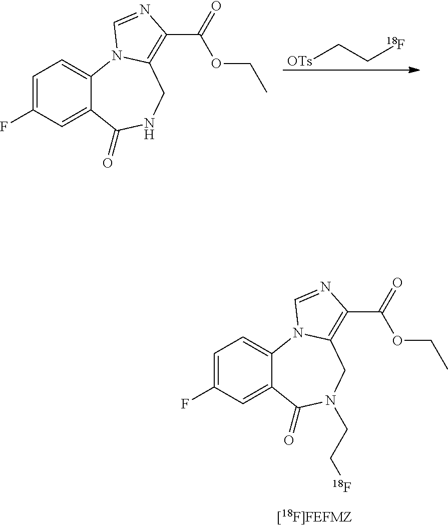

This synthesis of [¹⁸F]FEFMZ has been reported as high yielding. However, the clearance of this compound following administration in vivo is too rapid to enable in vivo imaging.

The present invention seeks to provide alternative radiofluorinated compounds suitable for studying the GABA_{A} receptor in vivo wherein said compounds have improved properties over those known in the prior art.

### SUMMARY OF THE INVENTION

The present invention provides novel radiofluorinated compounds useful for in vivo imaging GABA_{A} receptors. The synthesis of the radiofluorinated compounds of the invention is high-yielding. Also provided by the present invention is a method of synthesis for the radiofluorinated compounds of the invention, in particular an automated method of synthesis. A further aspect of the invention is a cassette suitable for carrying out the automated method of synthesis of the invention.

### DETAILED DESCRIPTION OF THE INVENTION

In one aspect the present invention relates to a radiofluorinated compound of Formula I:

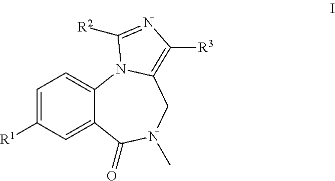

wherein:

one of R¹ or R² is C₁₋₄ [¹⁸F]fluoroalkyl or C₁₋₄ [¹⁸F]fluoroalkoxy, and the other is hydrogen; and,

R³ is C(═O)—O—R⁴ wherein R⁴ is hydrogen, or straight- or branched-chain C₁₋₄ alkyl; or,

R⁴ is a C₃₋₅ heterocycle.

The term “radiofluorinated compound” refers to a compound where the molecular formula comprises ¹⁸F. The ready availability and physical properties of ¹⁸F make it the radioisotope of choice in the development of PET radiotracers (Snyder and Kilbourn “Chemistry of Fluorine-18 Radiopharmaceuticals” pp 195-227; “Handbook of Radiopharmaceuticals” 2003: Welch and Redvanly, Eds).

Suitable salts according to the invention include (i) physiologically acceptable acid addition salts such as those derived from mineral acids, for example hydrochloric, hydrobromic, phosphoric, metaphosphoric, nitric and sulphuric acids, and those derived from organic acids, for example tartaric, trifluoroacetic, citric, malic, lactic, fumaric, benzoic, glycollic, gluconic, succinic, methanesulphonic, and para-toluenesulphonic acids; and (ii) physiologically acceptable base salts such as ammonium salts, alkali metal salts (for example those of sodium and potassium), alkaline earth metal salts (for example those of calcium and magnesium), salts with organic bases such as triethanolamine, N-methyl-D-glucamine, piperidine, pyridine, piperazine, and morpholine, and salts with amino acids such as arginine and lysine.

Suitable solvates according to the invention include those formed with ethanol, water, saline, physiological buffer and glycol.

The term “alkyl” means straight-chain or branched-chain alkyl radical containing preferably from 1 to 4 carbon atoms. Examples of such radicals include methyl, ethyl, and propyl.

The term “alkoxy” means an alkyl ether radical wherein the term alkyl is as defined above. Examples of suitable alkoxy groups include, methoxy, ethoxy, and propoxy.

The terms “[¹⁸F]fluoroalkyl” and “[¹⁸F]fluoroalkoxy” refer to alkyl and alkoxy groups, respectively, as defined above, substituted with ¹⁸F. Suitably, ¹⁸F replaces one of the hydrogens at the distal terminus of the substituent, i.e. C₁₋₄ [¹⁸F]fluoroalkyl is —(CH₂)ₙ—¹⁸F and C₁₋₄ [¹⁸F]fluoroalkoxy is —O—(CH₂)ₙ—¹⁸F, wherein n in both cases is 1-4.

The term “heterocycle” refers herein to an aliphatic or aromatic cyclic radical wherein the cycle comprises one or more heteroatoms selected from nitrogen, oxygen or sulfur.

In a preferred embodiment of the radiofluorinated compound of Formula I, one of R¹ and R² is C₁₋₄ [¹⁸F]fluoroalkyl, most preferably R¹. Preferred C₁₋₄ [¹⁸F]fluoroalkyl groups are [¹⁸F]fluoromethyl and [¹⁸F]2-fluoroethyl.

In a more preferred embodiment of the radiofluorinated compound of Formula I, one of R¹ and R² is C₁₋₄ [¹⁸F]fluoroalkoxy, most preferably R¹. Preferred C₁₋₄ [¹⁸F]fluoroalkoxy groups are [¹⁸F]fluoromethoxy and [¹⁸F]2-fluoroethoxy, most preferably [¹⁸F]fluoroethoxy.

A preferred R³ group of Formula I is C(═O)—O—R⁴ wherein R⁴ is straight- or branched-chain C₁₋₄ alkyl, most preferably methyl, ethyl or tent-butyl.

In another aspect, the present invention provides a method for the synthesis of a radiofluorinated compound of Formula I, wherein said method comprises reaction with a suitable source of ¹⁸F of a precursor compound of Formula Ia:

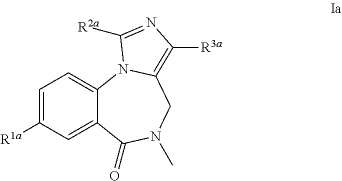

wherein:

one of R^{1a} and R^{2a} is a precursor group, and the other is H, wherein when R^{1a} is a precursor group it is selected from C₁₋₄ alkyl-LG, C₁₋₄ alkoxyl-LG and hydroxyl, and wherein when ^{Rea} is a precursor group it is selected from C₁₋₄ alkyl-LG and C₁₋₄ alkoxyl-LG, wherein LG is a leaving group selected from bromide, mesylate or tosylate; and,

R^{3a} is as defined for R³ of Formula I.

A “suitable source of ¹⁸F” means ¹⁸F in a chemical form that is reactive with a precursor group in the precursor compound such that the ¹⁸F becomes covalently attached, resulting in the radiofluorinated compound of Formula I. The choice of suitable source of ¹⁸F depends on the precursor group with which it is intended to react. Further discussion is provided below.

Broadly speaking, the step of “reacting” the precursor compound with the suitable source of ¹⁸F involves bringing the two reactants together under reaction conditions suitable for formation of the desired radiofluorinated compound in as high a radiochemical yield (RCY) as possible. Some detailed routes are provided below.

A “precursor compound” of the present invention comprises a non-radioactive derivative of the radiofluorinated compound of Formula I, comprising a precursor group at the desired location of the ¹⁸F label so that chemical reaction with a convenient chemical form of ¹⁸F occurs site-specifically. The precursor compound is designed so that radiofluorination can be conducted in the minimum number of steps (ideally a single step) and without the need for significant purification (ideally no further purification) to give the desired radiofluorinated compound of Formula I. Such precursor compounds are synthetic and can conveniently be obtained in good chemical purity. The precursor compound may be provided in solution in a kit, or in a cassette suitable for use with an automated synthesis apparatus. The kit and cassette form additional aspects of the invention and will be discussed in more detail below.

A “precursor group” is a substituent of the precursor compound as defined above which reacts with the source of ¹⁸F such that ¹⁸F is incorporated site-specifically to result in the desired radiofluorinated compound of Formula I.

A “leaving group” is an atom or group of atoms that is displaced as a stable species taking with it the bonding electrons. Suitable leaving groups in the context of the present invention include bromide, mesylate and tosylate.

The reaction scheme disclosed by Yang et al (2009 Synthesis; 6: 1036-1040) can be adapted to obtain precursor compounds wherein R^{1a} is a precursor group. Scheme 1 illustrates how the precursor compounds can be obtained:

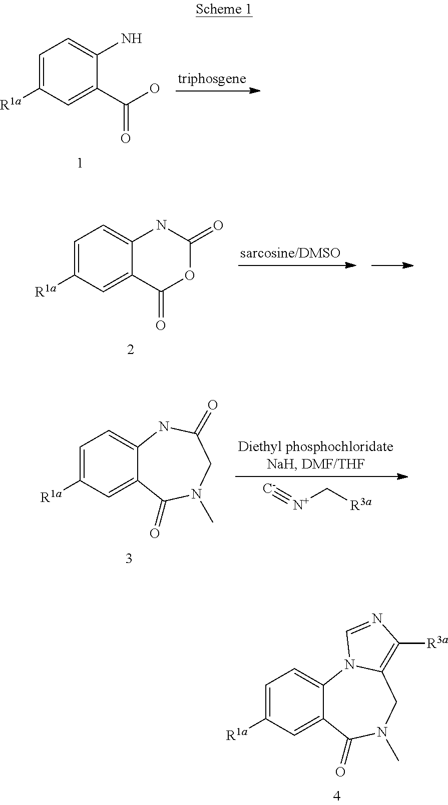

The appropriate amino benzoic acid compound (1), equipped to perform the required chemistry to introduce the desired leaving group at later stage, is reacted with triphosgene to afford the benzoxazine-2,4-dione intermediate (2). Reaction of 2 with sarcosine in DMSO yields the benzodiazepine (3). The compound of general structure 4 is obtained in good yields using the described conditions. 4 can be further modified to give the appropriate precursor compound using standard chemical transformations.

Example 2 below describes how to obtain the precursor compound “Precursor Compound 1” wherein Rⁱa is hydroxyl, ^{Rea} is hydrogen, and R^{3a} is C(═O)—O—R⁴ wherein R⁴ is ethyl. Example 4 below describes how to obtain the precursor compound “Precursor Compound 2” wherein R^{1a} is hydroxyl, R^{2a} is hydrogen, and R^{3a} is C(═O)—O—R⁴ wherein R⁴ is tent-butyl.

Where R² is a precursor group, the precursor compound can be obtained using the chemistry described in Scheme 2 below, where the appropriate isocyanate acetate is prepared using standard alkylation conditions from commercially available materials. Compound 8 can be appropriately modified using standard chemical transformations to generate the desired precursor.

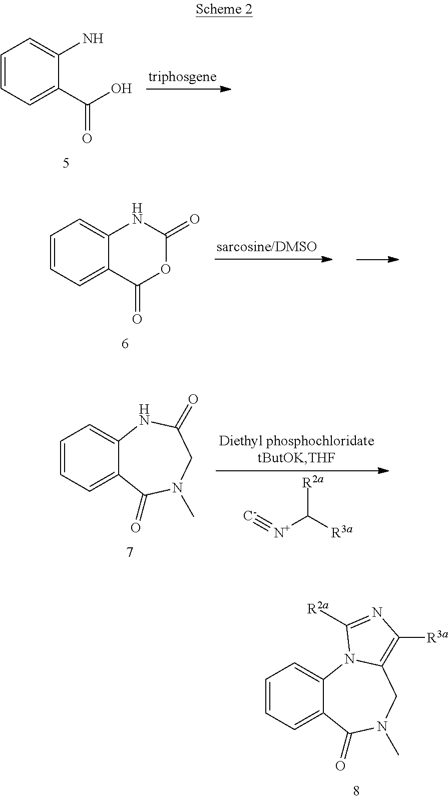

Precursor compounds of Formula Ia wherein Rⁱa comprises a heterocycle can be obtained by methods described by Watjen et al (J Med Chem 1989; 32(10): 2282-2291).

Introduction of ¹⁸F may be achieved via direct labelling comprising reaction of a precursor compound comprising a leaving group (LG), i.e. bromide, mesylate or tosylate, preferably tosylate, with ¹⁸F-fluoride as the suitable source of ¹⁸F. [¹⁸F]fluoride (¹⁸F⁻) for radiofluorination reactions is normally obtained as an aqueous solution from the nuclear reaction ¹⁸O(p,n)¹⁸F and is made reactive by the addition of a cationic counterion and the subsequent removal of water. Suitable cationic counterions should possess sufficient solubility within the anhydrous reaction solvent to maintain the solubility of ¹⁸F⁻. Therefore, counterions that have been used include large but soft metal ions such as rubidium or caesium, potassium complexed with a cryptand such as Kryptofix™, or tetraalkylammonium salts. A preferred counterion is potassium complexed with a cryptand such as Kryptofix™ because of its good solubility in anhydrous solvents and enhanced ⁸F⁻ reactivity. ¹⁸F⁻ that has been made reactive in this way, reacted with a precursor compound of Formula Ia comprising C₁₋₄ alkyl-LG or C₁₋₄ alkoxy-LG, results in a radiofluorinated compound of Formula I comprising C₁₋₄ [¹⁸F]-fluoroalkyl or C₁₋₄ [¹⁸F]-fluoroalkoxy. The alkyl or alkoxy in the C₁₋₄ alkyl-LG or C₁₋₄ alkoxy-LG correspond to the alkyl or the alkoxy in the C₁₋₄ [¹⁸F]-fluoroalkyl or C₁₋₄ [¹⁸F]-fluoroalkoxy, respectively, wherein C₁₋₄ [¹⁸F]-fluoroalkyl or C₁₋₄ [¹⁸F]-fluoroalkoxy are as suitably and preferably defined above for Formula I. Suitable and preferred leaving groups LG are as defined above.

¹⁸F can also be introduced by O-alkylation of hydroxyl groups in the precursor compound with a synthon comprising ¹⁸F, e.g. fluoroalkyl bromide, [¹⁸F]-fluoroalkyl mesylate or [¹⁸F]-fluoroalkyl tosylate. Therefore, a precursor compound of Formula Ia where the precursor group of R^{1a} is hydroxyl, is reacted with C₁₋₄ [¹⁸F]-fluoroalkyl-LG as the suitable source of ¹⁸F to obtain a radiofluorinated compound of Formula I comprising C₁₋₄ [¹⁸F]-fluoroalkoxy.

Example 2(iii) describes the radiofluorination of Precusor Compound 1, which comprises a hydroxyl precursor group, with [¹⁸F]-fluoroethyltosylate to obtain [¹⁸F]-Compound 1. The Kⁱ of non-radioactive Compound 1 was found to be 2.4 nM (see Example 5). Biodistribution of [¹⁸F]-Compound 1 in an in vivo model showed good regional differentiation, i.e. between GABA-rich and GABA-poor regions of the brain (see Example 6).

Example 4(v) describes the radiofluorination of Precusor Compound 2, which also comprises a hydroxyl precursor group, with ^{[}¹⁸F] -fluoroethyltosylate to obtain [¹⁸F]-Compound 2. The Kᵢ of non-radioactive Compound 2 was found to be 0.53 nM (see Example 5). Biodistribution of [¹⁸F]-Compound 2in an in vivo model showed good regional differentiation, i.e. between GABA-rich and GABA-poor regions of the brain (see Example 7).

In a preferred embodiment of the method of the invention, R^{1a} of the precursor compound of Formula Ia is a precursor group. When R^{1a} is a precursor group, it is preferably C₁₋₄ alkoxy-LG or hydroxyl, especially preferably methoxy-LG, ethoxy-LG or hydroxyl, and most especially preferably hydroxyl.

The synthesis of ¹⁸F-labelled compounds, particularly for use as PET tracers, is currently most conveniently carried out by means of an automated synthesis apparatus, e.g. Tracerlab™ and Fastlab™ (both GE Healthcare). Fastlab™ represents the state of the art in automated PET radiotracer synthesis platforms, so that it is desirable in the development of a new PET radiotracer that its synthesis is compatible with Fastlab™ The radiofluorinated compounds of the present invention are advantageous over those of the prior art in this respect as their synthesis is Fastlab™-compatible. Therefore, in a preferred embodiment, the method of the invention is automated. The radiochemistry is performed on the automated synthesis apparatus by fitting a “cassette” to the apparatus. Such a cassette normally includes fluid pathways, a reaction vessel, and ports for receiving reagent vials as well as any solid-phase extraction cartridges used in post-radiosynthetic clean up steps.

In a further aspect of the present invention there is provided a cassette for carrying out the automated method of the invention comprising:

(i) a vessel containing a precursor compound, wherein said precursor compound is as defined above for the method of the invention; and

(ii) means for eluting the vessel with a suitable source of ¹⁸F, wherein said suitable source of ¹⁸F is as defined above for the method of the invention.

The cassette may also comprise an ion-exchange cartridge for removal of excess ¹⁸F. The reagents, solvents and other consumables required for the automated synthesis may also be included together with a data medium, such as a compact disc carrying software, which allows the automated synthesiser to be operated in a way to meet the end user's requirements for concentration, volumes, time of delivery etc.

Also provided by the present invention is a “radiopharmaceutical composition”, which comprises the radiofluorinated compound as defined herein together with a biocompatible carrier in a form suitable for mammalian administration.

The “biocompatible carrier” is a fluid, especially a liquid, in which the radiofluorinated compound is suspended or dissolved, such that the radiopharmaceutical composition is physiologically tolerable, i.e. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). The biocompatible carrier may also comprise biocompatible organic solvents such as ethanol. Such organic solvents are useful to solubilise more lipophilic compounds or formulations. Preferably the biocompatible carrier is pyrogen-free water for injection, isotonic saline or an aqueous ethanol solution. The pH of the biocompatible carrier for intravenous injection is suitably in the range 4.0 to 10.5.

Suitable and preferred embodiments of the radiofluorinated compound when comprised in the radiopharmaceutical composition of the invention are as already described herein.

The radiopharmaceutical composition may be administered parenterally, i.e. by injection, and is most preferably an aqueous solution. Such a composition may optionally contain further ingredients such as buffers; pharmaceutically acceptable solubilisers (e.g. cyclodextrins or surfactants such as Pluronic, Tween or phospholipids); pharmaceutically acceptable stabilisers or antioxidants (such as ascorbic acid, gentisic acid or para-aminobenzoic acid). Where the radiofluorinated compound of the invention is provided as a radiopharmaceutical composition, the method for preparation of said radiofluorinated compound may further comprise the steps required to obtain a radiopharmaceutical composition, e.g. removal of organic solvent, addition of a biocompatible buffer and any optional further ingredients. For parenteral administration, steps to ensure that the radiopharmaceutical composition is sterile and apyrogenic also need to be taken.

The present invention provides in a further aspect the radiofluorinated compound as suitably and preferably defined herein for use in a method of in vivo imaging. Most preferably the radiofluorinated compound for use in a method of in vivo imaging is provided as the radiopharmaceutical composition as suitably and preferably defined herein.

In a yet further aspect, the present invention provides a positron emission tomography (PET) method for determining the distribution of GABA_{A} receptors in the central nervous system (CNS) of a subject comprising:

(i) administering to said subject the radiofluorinated compound as suitably and preferably defined herein;

(ii) allowing said administered radiofluorinated compound of step (i) to bind to GABA_{A} receptors in the CNS of said subject;

(iii) detecting signals derived from the positron emission decay of the ¹⁸F present in said bound radiofluorinated compound of step (ii); and,

(iv) generating an image of the location and amount of said signals, wherein said signals represent the distribution of GABA_{A} receptors in said subject.

For the PET method of the invention, suitable and preferred aspects of the radiofluorinated compound are as defined earlier in the specification.

“Administering” the radiofluorinated compound is preferably carried out parenterally, and most preferably intravenously. The intravenous route represents the most efficient way to deliver the radiofluorinated compound throughout the body of the subject, and therefore also across the blood-brain barrier (BBB) and into contact with GABA_{A} receptors expressed in the CNS of said subject. The radiofluorinated compound of the invention is preferably administered as the radiopharmaceutical composition of the invention, as defined herein.

Following the administering step and preceding the detecting step, the radiofluorinated compound is allowed to bind to GABA_{A} receptors. The radiofluorinated compound moves dynamically through the mammal's body, coming into contact with various tissues therein.

Once the radiofluorinated compound comes into contact with GABA_{A} receptors, a specific interaction takes place such that clearance of the radiofluorinated compound from tissue with GABA_{A} receptors takes longer than from tissue without, or having less GABA_{A} receptors. A certain point in time is reached when detection of radiofluorinated compound specifically bound to GABA_{A} receptors is enabled as a result of the ratio between radiofluorinated compound bound to tissue with GABA_{A} receptors versus that bound in tissue without, or having less GABA_{A} receptors. Ideally, this ratio is 2:1 or greater.

The “detecting” step of the method of the invention involves detection of signals derived from the positron emission decay of ¹⁸F by means of a detector sensitive to said signals, a scintillator present in the PET scanner. In positron-emission decay, which is also known as positive beta decay, a positron is emitted, and then travels up to a few millimetres until it encounters an electron. The encounter of the positron and the electron results in the production of a pair of annihilation (gamma) photons that are emitted at around 180 degrees to each other. It is these annihilation photons that are the “signals derived from the positron emission decay”.

The “generating” step of the method of the invention is carried out by a computer which applies a reconstruction algorithm to the acquired signal data to yield a dataset. This dataset is then manipulated to generate an image showing the location and/or amount of signals emitted by ¹⁸F.

The “subject” of the invention can be any human or animal subject. Preferably the subject of the invention is a mammal. Most preferably, said subject is an intact mammalian body in vivo. In an especially preferred embodiment, the subject of the invention is a human.

The PET method may be used to study GABA_{A} receptors in healthy subjects, or in subjects known or suspected to have a pathological condition associated with abnormal expression of GABA_{A} receptors (a “GABA_{A} condition”). Examples of such GABA_{A} conditions where the PET method of the invention would be of use include epilepsy, anxiety disorders, Parkinson's disease and chronic pain. The radiofluorinated compound of the invention is particularly suited to PET imaging GABA_{A} receptor expression in the central nervous system (CNS).

In an alternative embodiment, the PET method of the invention may be carried out repeatedly during the course of a treatment regimen for said subject, said treatment regimen comprising administration of a drug to combat a GABA_{A} condition. For example, the PET method as suitably and preferably defined herein can be carried out before, during and after treatment with a drug to combat a GABA_{A} condition. In this way, the effect of said treatment can be monitored over time. PET has excellent sensitivity and resolution, so that even relatively small changes in a lesion can be observed over time, which is advantageous for treatment monitoring. PET scanners routinely measure radioactivity concentrations in the picomolar range. Micro-PET scanners now approach a spatial resolution of about 1 mm, and clinical scanners about 4-5 mm.

In a further aspect, the present invention provides a method for the diagnosis of a GABA_{A} condition. The method of diagnosis of the invention comprises the PET method as suitably and preferably defined above, together with the further step (v) of attributing the distribution of GABA_{A} expression to a particular clinical picture, i.e. the deductive medical decision phase.

In another aspect, the present invention provides the radiofluorinated compound as suitably and preferably defined herein for use in the method of diagnosis as defined herein.

In a yet further aspect, the present invention provides the in vivo imaging agent as defined herein for use in the manufacture of a radiopharmaceutical composition as defined herein for use in the method of diagnosis as defined herein.

The invention is now illustrated by a series of non-limiting examples.

### BRIEF DESCRIPTION OF THE EXAMPLES

Example 1 describes a method for the synthesis of non-radioactive Compound 1.

Example 2 describes a method for the synthesis of radiofluorinated Compound 1 from Precursor Compound 1.

Example 3 describes a method for the synthesis of non-radioactive Compound 2.

Example 4 describes a method for the synthesis of radiofluorinated Compound 2 from Precursor Compound 2.

Example 5 describes an in vitro assay that was used to evaluate the affinity of non-radioactive Compound 1 and Compound 2 for GABA_{A} receptors.

Examples 6 and 7 describe the in vivo biodistribution of [¹⁸F]-Compound 1 and [¹⁸F]-Compound 2, respectively.

Comparative example 8 describes a known method to obtain [¹⁸F]-flumazenil.

### List of Abbreviations used in the Examples

% id percentage of injected dose

% id/g percentage of injected dose per gram

DCM dichloromethane

DMSO dimethylsulfoxide

FFMZ fluoroflumazenil

FEFMZ fluoroethylflumazenil

FMZ flumazenil

MBq megabequerel(s)

OTs tosylate

pi post-injection

SD standard deviation

THF tetrahydrofuran

### Examples

### Example 1: Synthesis of Non-radioactive Compound 1

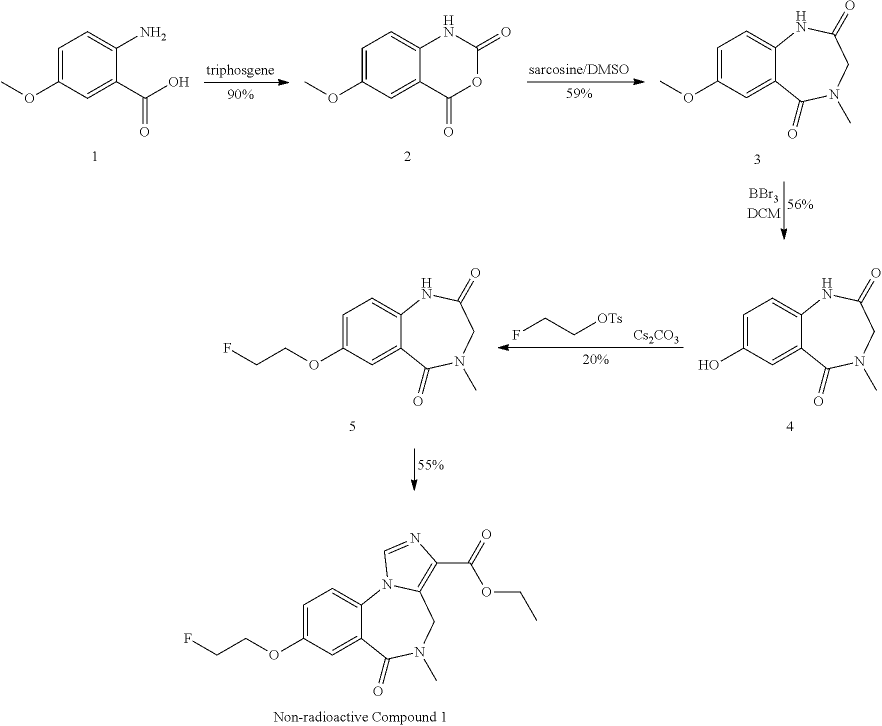

### Example 1(i): Synthesis of 6-Methoxy-1H-benzo [d] [1,3]oxazine-2,4-dione (2)

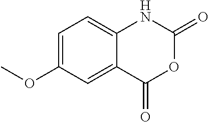

Commercially-available 2-Amino-5-methoxybenzoic acid (20 g, 120 mmol) was dissolved in dioxane (200 mL). Triphosgene (15 g, 50.6 mmol) was added with cooling (during the addition a thick precipitate formed). Dioxane (50 mL) was added to aid mobility. The mixture was heated under reflux for 1 h and then allowed to cool. The resulting precipitate was collected by filtration to afford intermediate 2 as a beige powder (20.8 g, 90%).

¹H NMR (D₆-DMSO): δ 3.81 (3H, s, CH₃), 7.11 (1H, d, J=9 Hz, NHCCHCHCOCH₃), 7.34 (1H, d, J=3 Hz, CH₃OCCHCCO), 7.39 (1H, dd, J=9 and 3 Hz, CHCOCH₃CH), 11.6 (1H, br s, NH).

### Example 1 (ii): Synthesis of 7-Methoxy-4-methyl-3,4-dihydro-1H-benzo[e] [1,4]diazepine-2,5-dione (3)

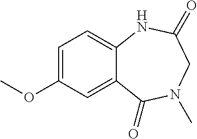

Intermediate 2 (20.8 g, 108 mmol) was suspended in DMSO (55 mL). The mixture was then placed on a preheated mantle (157° C.). The mixture was stirred. Once almost all of the starting material was in solution sarcosine (32.0 g, 108 mmol) was added portionwise. Almost immediately effervescence was observed. The mixture was heated for 2 h after which time the mixture was allowed to cool to ca. 70° C., and then poured into water (300 mL). Small white baubles were seen to form which then expanded to form a white powder. This was collected by filtration and then dried in a vacuum oven overnight at 50° C. (13.9 g, 59%).

¹H NMR (D₆-DMSO) δ 3.14 (3H, s, NCH₃), 3.75 (3H, s, OCH₃), 3.82 (2H, s, NCH₂), 7.03 (1H, d, J=9 Hz, CHCHCOCH₃), 7.12 (1H, dd, J=9 and 3 Hz, CHCHCOCH₃), 7.22 (1H, d, J=3 Hz, COCCHCOCH₃), 10.3 (1H, br s, NH).

Example 1 (iii): Synthesis of 7-Hydroxy-4-methyl-3,4-dihydro-1H-benzo[e] [1,4]diazepine-2,5-dione (4)

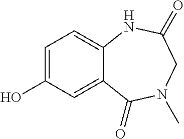

Boron tribromide (1M in DCM) (6.8 mL, 6.81 mmol) was added dropwise to a stirred suspension of intermediate 3 (0.5 g, 2.27 mmol) in anhydrous DCM (10 mL) (under a flow of nitrogen and at −78 C). Once addition was complete the mixture was allowed to stir at room temperature under nitrogen for 16 h. The solvent was then removed in vacuo and ice water carefully poured into the residue. The insoluble material was then collected by filtration and found to be the desired product (0.2 g, 43%).

¹H NMR (D₆-DMSO) δ 3.08 (3H, s, NCH₃), 3.70-3.80 (2H, m, NCH₂), 6.91 (2H, s, ArCH×2), 7.10 (1H, s, ArCH), 10.2 (1H, br s, NH)

### Example 1(iv): Synthesis of 7-(2-Fluoro-ethoxy)-4-methyl-3,4-dihydro-1H-benzo[e] [1,4]diazepine-2,5-dione (5)

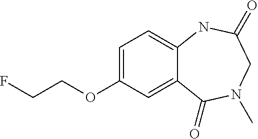

Cesium carbonate (8.0 g, 24.6 mmol) was added to intermediate 4 (3.4 g, 16.4 mmol) and fluoroethyl tosylate (5.4 g, 24.6 mmol) in DMF (100 mL). The mixture was heated at 60 C for 2 h (during which time the mixture had become dark brown). TLC (90% DCM, 10% MeOH) indicated that the reaction was complete. The solvent was removed under reduced pressure and the residue was then washed with water and organics extracted with ethyl acetate. The organic phase was then dried over MgSO₄, filtered and evaporated to dryness to afford the crude product. This was then purified by flash chromatography 100% DCM-95% DCM, 5% Me0H to afford the desired product (0.8 g, 20%).

¹H NMR (D₆-DMSO) δ 3.11 (3H, s, NCH₃), 3.82 (2H, s, NCH₂), 4.25 (2H, dt, J_{HF}=30 Hz, J_{HH} =4 Hz, CH₂O), 4.74 (2H, dt, J_{HF}=48 Hz, J_{HH}=4 Hz, CH₂F), 7.04 (1H, d, J=9 Hz, CHCHCOCH₂CH₂F), 7.16 (1H, dd, J=9 and 3 Hz, CHCHCOCH₂CH₂F), 7.24 (1H, d, J=3 Hz, COCCHCOCH₂CH₂F), 10.30 (1H, br s, NH).

### Example 1(v): Synthesis of Non-radioactive Compound 1

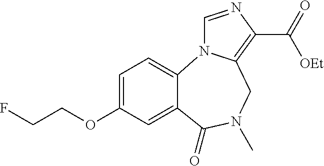

Intermediate 5 (0.80 g, 3.17 mmol) was suspended in DMF (6 mL) and THF (10 mL). Sodium hydride (0.15 g of a 60% dispersion in mineral oil, 3.79 mmol) was then added with cooling under nitrogen. After hydrogen evolution had ceased diethyl phosphorochloridate (0.67 mL, 4.75 mmol) was then added dropwise with cooling (the solution became bright yellow). Directly after, a solution of ethyl isocyanoacetate (0.41 mL, 3.80 mmol) in DMF (3 mL) was prepared under N₂. Sodium hydride (0.11 g of a 60% dispersion in mineral oil, 4.58 mmol) was then added with cooling. After hydrogen evolution had ceased the mixture was added dropwise to intermediate 5 with cooling. The mixture was stirred at 0° C. for 30 minutes and left to stir at room temperature for 18 h. Acetic acid (0.17 mL, 6.14 mmol) was then added to the reaction. The mixture was then poured into ice water and the organic material was extracted with ethyl acetate, dried over MgSO₄, filtered and evaporated to dryness. The resulting brown oil was then subjected to flash chromatography twice using DCM 100%->95% DCM, MeOH 5%. The resulting bright yellow solid was then washed with ether until the ether remained colourless. The pale yellow solid was collected by filtration (0.6 g, 55%).

¹H NMR (CDCl₃) δ 1.44 (3H, s, CH₃), 3.24 (3H, s, NCH₃), 4.19-4.45 (5H, m, OCH₂, NCH, OCH₂), 4.78 (2H, dt, J_{HF}=47 Hz, J_{HH}=4 Hz, CH₂F), 5.20 (1H, br s, NCH′), 7.21 (1H, dd, J=9 and 3 Hz, CHCHCOCH₂CH₂F), 7.36 (1H, d, J=8 Hz, CHCHCOCH₂CH₂F), 7.54 (1H, d, J=3 Hz, COCCHCOCH₂CH₂F), 7.84 (1H, s, NCHN).

### Example 2: Synthesis of Radio fluorinated Compound 1

### Example 2(i): Synthesis of 8-Methoxy-5-methyl-6-oxo-5,6-dihydro-4H-2,5,10b-triaza-benzo[e]azulene-3-carboxylic acid ethyl ester (6)

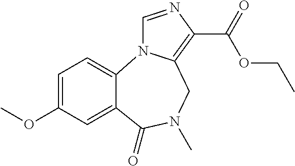

Intermediate 3 (1.0 g, 4.54 mmol; synthesis described in Example 1(ii)) was suspended in DMF (9 mL) and THF (14 mL). Sodium hydride (0.13 g of a 60% dispersion in mineral oil, 5.41 mmol) was then added with cooling under nitrogen. After hydrogen evolution had ceased diethyl phosphorochloridate (1.18g, 0.99 mL, 6.81 nmmol) was then added dropwise with cooling (the solution became bright yellow). Directly after, a solution of ethyl isocyanoacetate (0.62 g, 0.60 mL, 5.48 mmol) in DMF (4.5 mL) was prepared under N₂. Sodium hydride was then added (0.15 g of a 60% dispersion in mineral oil, 6.25 mmol) with cooling. After hydrogen evolution had ceased the mixture was added dropwise to intermediate 3 with cooling. The mixture was an orange suspension. The mixture was left to stir at room temperature for 18 h. Acetic acid (1 mL) was then added to the reaction. The mixture was then poured into ice water. A precipitate was observed. This was collected by filtration and washed with water, dried and then washed with diethyl ether. The solid was found to be pure product (0.58 g). The aqueous filtrate was washed with ethyl acetate, dried over MgSO₄, filtered and evaporated to dryness. The resulting orange solid was then was then washed with ether. The pale yellow solid was then collected by filtration (0.2 g+0.58 g=57%).

¹H NMR (CDCl₃) δ 1.45 (3H, s, CH₃), 3.25 (3H, s, NCH₃), 3.91 (3H, s, OCH₃), 4.25-4.49 (3H, m, OCH₂, NCH), 5.16-5.21 (1H, m, NCH′), 7.13 (1H, dd, J=9 and 3 Hz, CHCHCOCH₃), 7.35 (1H, d, J=9 Hz, CHCHCOCH₃), 7.55 (1H, d, J=3 Hz, COCCHCOCH₃), 7.84 (1H, s, NCHN).

### Example 2(h): Synthesis of Precursor Compound 1

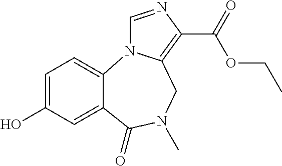

Intermediate 6 (0.55 g, 1.74 mmol) was dissolved in DCM (5 mL), boron tribromide (1.75 mL of a 1M solution in dichloromethane, 1.75 mmol) was then added dropwise at −70° C. After 1 h a sample was then taken from the mixture and diluted with methanol. TLC (95% DCM, 5% MeOH) indicated presence of starting material and new spot on baseline. An NMR of this sample showed it was the H-salt of the imidazole and no demethylation had yet taken place. The reaction was left in the freezer overnight. The following day another equivalent of boron tribromide was added dropwise at −70 C. After 1 h TLC indicated the presence of starting material, baseline material and a new spot just below the starting material. The mixture was left to stir at room temperature for 3 h. TLC indicated most of the starting material had gone and LCMS indicated the presence of product. Another NMR sample was taken by diluting with methanol evaporating and redissolving in deuterated methanol. The NMR indicated the presence of 4 compounds, two of which where methyl esters. This indicated that hydrolysis of the ester was occurring during the BBr₃ reaction to give the carboxylic acid which was then methylated during methanol work-up. Therefore more product could be obtained by re-esterifying in situ: the bulk reaction mixture was therefore diluted with ethanol (slowly with caution!). The reaction mixture was slightly warm after this addition and then left to stir over the weekend at RT. The mixture was then evaporated and dissolved in water and neutralised. The aqueous phase was then washed with ethyl acetate and the organic phases were then combined and dried over MgSO₄, filtered and evaporated to dryness to form an orange solid. This was washed with ether until the ether was colourless. The solid was then subjected to column chromatography using 99% DCM, 1% MeOH→3% MeOH . Product eluted at 30 CV. Impurity removed at 5 CV (1% MeOH). Solid load on silica, 4 g column. The desired product was obtained as a white solid (20 mg, 4%).

¹H NMR (D₃-Methanol) δ 1.41 (3H, s, CH₃), 3.20 (3H, s, NCH₃), 4.32-4.55 (3H, m, NCH, OCH₂), 5.12 (1H, br d, J=15 Hz, NCH′), 7.11 (1H, dd, J=9 Hz and 3 Hz, CHCHCOH), 7.34 (1H, d, J=3 Hz, OCCCHCOH), 7.51 (1H, d, J=9 Hz, CHCHCOHCH), 8.18 (1H, s, NCHN).

### Example 2(iii): Radiofluorination to obtain [¹⁸F]-Compound 1

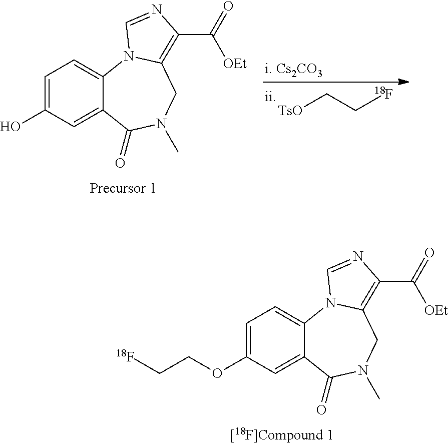

[¹⁸F]fluoride was transferred from a P6 vial into a 3mL V-vial by suction. To the P6 vial was added a pre-prepared solution of Kryptofix 2.2.2 (4 mg) in MeCN (0.5 mL) and KHCO₃ (100 μL, 0.1M). The vial was agitated and the solution transferred to the V-vial by suction. The vial was heated to 110° C. for 20 min under a flow of nitrogen (0.2 L/min) then cooled to room temperature. To the dried [¹⁸F]fluoride and Kryptofix 2.2.2 mixture was added ethanediol-p-toluenesulfonate (5 mg) in MeCN (1 mL). The resulting yellow solution was heated at 80° C. for 10 min, and then cooled to room temperature. To the reaction vial was added water (1.5 mL) and loaded on to preparative HPLC for purification (Hichrom ACE C5 10×100mm column; solvent A=50 mM Ammonium Acetate, solvent B=MeCN; flow rate 4 mL/min; UV 254 nm). The isolated HPLC fraction was diluted into water (20 mL) and then loaded onto a Waters tC 18-light Sep Pak cartridge. The cartridge was then dried on a high pressure nitrogen line for 15 min.

Precursor Compound 1 (2 mg) and caesium carbonate (10 mg) were carefully weighed into a 1 mL Wheaton vial then DMF (0.1 mL) was added along with a stirrer bar. The suspension was stirred at room temperature for 10 min. The dried [¹⁸F]fluoroethyltosylate was eluted off the SPE into the Wheaton vial with DMF (0.5 mL) and the resulting reaction mixture was stirred at 130° C. for 10 min. The reaction mixture was cooled and was diluted into 50 mM ammonium acetate (3.5 mL) and then purified by HPLC (Hichrom ACE C5 10×100 mm column; solvent A=50 mM Ammonium Acetate, solvent B=MeCN; flow rate 4 mL/min; UV 254 nm).

The isolated HPLC fraction was diluted into water (20 mL) and trapped onto a tC18light Sep Pak and then eluted with ethanol (0.5 mL) into a pre-weighed vial containing PBS (0.5 mL). The ethanol was removed in vacuo until the original mass was obtained. An aliquot (50 MBq) of [¹⁸F]Compound 1 was formulated in PBS at 5 MBq/mL for use in the in vivo biodistribution assay described in Example 6 below.

Analytical HPLC (Phenomenex Luna C18(2) 50×2 mm column; solvent A=0.01M Phosphoric Acid, solvent B=MeCN; 0.4 mL/min; UV 254 nm) confirmed that [¹⁸F] Compound 1 was obtained at 95% radiochemical purity with an end of synthesis yield of 23%.

### Example 3: Synthesis of Non-radioactive Compound 2

### Example 3(i): Synthesis of 7-Bromo-4-methyl-3,4-dihydro-1H-benzo[e][1,4]diazepine-2,5-dione (7)

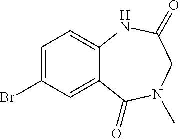

A mixture of 5-bromoisatoic anhydride (40.0 g, 165 mmol) and sarcosine (14.7 g, 165 mmol) in DMSO (100 mL) was placed in a heating mantle, which had been preheated to 148-150 C. Within a few moments the dark orange solution turned a pale orange and effervescence was observed. The mixture was heated at 150 C for ca. 30 min and then poured into water (600 mL). The resulting pale yellow precipitate was collected by filtration to afford 33.4 g (75%) of 7.

¹H NMR (300 MHz, DMSO-d₆): δ_{H} 3.11 (3H, s, NCH₃), 3.89 (2H, s, CH₂), 7.06 (1H, d, J=9.0 Hz, NHCCHCH), 7.69 (1H, dd, J=9.0 and 2.0 Hz, BrCHCH), 7.82 (1H, d, J=2.0 Hz, OCCCH), and 10.6 (1H, br s, NH).

### Example 3(h): Synthesis of Non-radioactive Compound 2

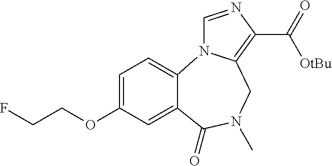

Potassium tert-butoxide (0.32 g, 2.83 mmol) was added to 7 (0.65 g, 2.58 mmol) in THF (52 mL) at 0 C. The mixture was then stirred at 0 C for 20 min (during which time a bright yellow precipitate was observed) and then cooled to −35 C. Diethyl chlorophosphate (0.58 g, 3.35 mmol, 0.49 mL) was added slowly. The reaction was stirred at 0 C for 30 min during which time mixture became bright yellow in colour. The reaction flask was cooled to −35 C and solution of tent-butyl isocyanoacetate (0.4 g, 2.83 mmol, 0.41 mL) was added followed by potassium tert-butoxide (0.32 g, 2.83 mmol). The suspension was then left to stir at room temperature overnight. The reaction was quenched with aq. NaHCO₃ (70 mL) and extracted with EtOAc (3×70 mL). The combined organic layers were dried over MgSO₄, concentrated to afford an orange syrup. The crude material was purified by silica gel chromatography eluting with DCM (A): MeOH (B) (1-5% B, 9 CV, 120 g, 40 mL/min). Non-radioactive Compound 2 was obtained as a pale yellow solid 0.53 g (55%).

¹H NMR (300 MHz, CDCl₃): δ_{H} 1.65 (9H, s, C(CH₃)₃), 3.25 (3H, s, NCH₃), 4.23-4.41 (5H, m, OCH₂, CONCH₃CHₐH_{b}), 4.80 (2H, dt, J_{HF}=47.0 and J=4.0 Hz, CH₂F), 5.15 (1H, br d, J=14.0 Hz, CONCH₃CHₐH_{b}), 7.21 (1H, dd, J=9.0 And 3.0 Hz, CHCHCOCH₂CH₂F), 7.36 (1H, d, J=9.0 Hz, NCCHCH), 7.55 (1H, d, J=3.0 Hz, OC—CCH), and 7.84 (1H, s, NCHN).

### Example 4: Synthesis of Radio fluorinated Compound 2

### Example 4(i): Synthesis of 7-benzyloxy-4-methyl-3,4-dihydro-1H-benzo[e] [1,4]diazepine-2,5-dione (8)

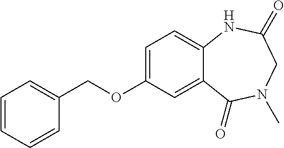

Cesium carbonate (6.53 g, 20 mmol) was added to 4 (4.13 g, 20 mmol, prepared according to Example 1(iii)) and benzyl bromide (3.42 g, 20 mmol, 2.38 mL) in DMF (50 mL). The mixture was heated at 60 C for 2 h. After which time TLC (90% DCM, 10% MeOH) indicated that the reaction was not complete. Another equivalent of benzyl bromide was added, after 1 h TLC indicated that the reaction was complete. The solvent was removed under reduced pressure the residue was then washed with water and ethyl acetate. A white precipitate was observed between the solvent interfaces this was collected by filtration. The organic phase was then dried over MgSO₄, filtered and evaporated to dryness to afford the crude product. This was triturated with a small amount of ethyl acetate and collected by filtration to give 8 As a white solid 2.77 g (47%).

¹H NMR (300 MHz, DMSO-d₆): δ_{H} 3.11(3H, s, NCH₃), 3.82 (2H, s, NCH₂), 5.12 (2H, s, OCH₂), 7.04 (1H, d, J=9.0 Hz, HNCCHCH), 7.19 (1H, dd, J=9.0 and 3.0 Hz, BnOCCHₐH_{b}), 7.31 (1H, d, J=3.0 Hz, O═CCCH), 7.33-7.47 (5H, m, CH×5), and 10.30 (1H, br s, NH).

### Example 4(h): Synthesis of 8-Benzyloxy-5-methyl-6-oxo-5,6-dihydro-4H-2,5,10b-triaza-benzo[e]azulene-3-carboxylic acid tert-butyl ester (9)

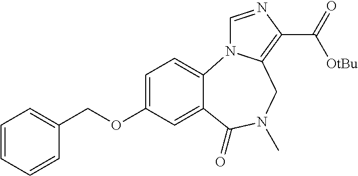

8 (2.7 g, 9.11 mmol) was suspended in DMF (24 mL) and THF (38 mL). Sodium hydride (0.43 g of a 60% dispersion in mineral oil, 10.8 mmol) was then added with cooling under nitrogen. After hydrogen evolution had ceased, diethyl chlorophosphate (2.36 g, 13.7 mmol, 1.98 mL) was then added dropwise with cooling (the solution became yellow). Directly after a solution of tert-butyl isocyanoacetate (1.54 g, 10.9 mmol, 1.59 mL) in DMF (12 mL) was prepared under N₂. Sodium hydride (0.51 g of a 60% dispersion in mineral oil, 12.9 mmol) was added with cooling. After hydrogen evolution had ceased the mixture was added dropwise to the 8 mixture with cooling. The mixture was an orange suspension. The mixture was left to stir at room temperature for 18 h. Acetic acid (1 mL) was then added to the reaction. The mixture was then poured into ice water and the organic material was extracted with ethyl acetate, dried over MgSO₄, filtered and evaporated to dryness. The crude material was purified by silica gel chromatography eluting with DCM (A): MeOH (B) (0-5% B, 10 CV, 50 g, 40 mL/min). The product was dissolved in minimal ethyl acetate then petroleum spirit was added dropwise until mixture became opaque. A few drops of ethyl acetate were added until solution became clear. The mixture was then left to stand for a couple of hours to afford 9 as a white solid 0.18 g (5%).

¹H NMR (300 MHz, CDCl₃): δ_{H} 1.69 (3H, s, 3×CH₃), 3.24 (3H, s, NCH₃), 4.36 (1H, br s, CONCH₃CHₐH_{b}), 5.05-5.16 (3H, m, OCH₂, CONCH₃CHₐH_{b}), 7.20 (1H, dd, J=9.0 and 3.0 Hz, CHCHCOBn), 7.32 (1H, d, J=9.0 Hz, NCCHCH), 7.35-7.46 (5H, m, ArCH×5), 7.63 (1H, d, J=3.0 Hz, OCCCH), and 7.81 (1H, s, NCHN).

### Example 4(iii): Synthesis of 8-Hydroxy-5-methyl-6-oxo-5,6-dihydro-4H-2,5,10b-triaza-benzo [e]azulene-3-carboxylic acid tent-butyl ester (Precursor Compound 2)

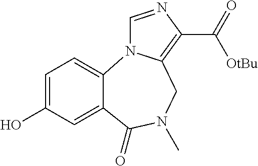

9 (50 mg, 0.36 mmol) was dissolved in methanol (10 mL). The mixture was then passed through a palladium cartridge (flow rate of 1 ml/min) and subjected to hydrogen flow full H₂ mode at 60 C. TLC indicated that the reaction was complete. The solution was evaporated to dryness to afford Precursor Compound 2 as a white solid (30 mg, 77%).

¹H NMR (300 MHz, DMSO-d₆): δ_{H} 1.56 (3H, s, C(CH₃)₃), 3.09 (3H, s, NCH₃), 4.42 (1H, br s, CONCH₃CHₐH_{b}), 4.85 (1H, br s, CONCH₃CHₐH_{b}), 7.09 (1H, dd, J=9.0 and 3.0 Hz, CHCHCOH), 7.25 (1H, d, J=3.0 Hz, OC—CCHCOH), 7.53 (1H, d, J=9.0 Hz, NCCHCH), and 8.22 (1H, s, NCHN).

### Example 4(v): Radiofluorination to obtain [¹⁸ F] Compound 2

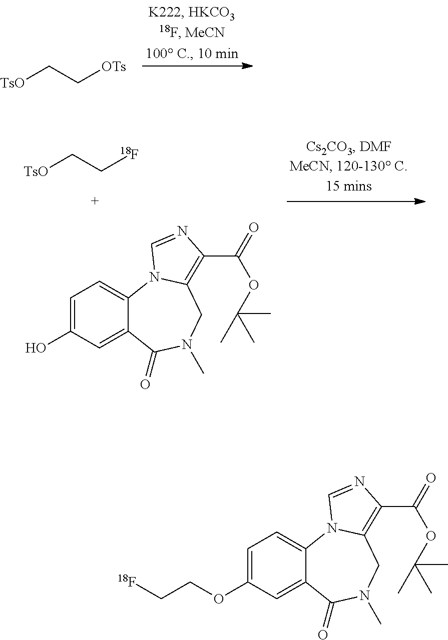

[¹⁸F]fluoride was drawn into a FAST1ab reaction vessel followed by Kryptofix 2.2.2 (2 mg) in acetonitrile (500 μl), KHCO₃ (0.1 mol dm⁻³, 50 ul) through the dip tube inlet. One nitrogen gas line was connected to the 2nd short inlet and a 2nd nitrogen gas line was connected to the closed dip tube valve. The nitrogen gas flow rate was set at 0.2-0.4 L/min. The heater controller was set at 100° C. Once this was reached, the ¹⁸F⁻ was dried for 5 minutes. After 5 minutes, the nitrogen gas flow was reduced to less than 0.1-0.2 L/min and the dip valve was opened and heated for a further 4 minutes. After 4 minutes, the nitrogen gas flow rate was increased to 0.2-0.4 L/min and dried for a further 11-16 minutes.

TsO-Et-OTs (5 mg) in acetonitrile (1000 μl) was added through the dip tube valve. The reaction vessel was sealed, the controller was set at 100° C. and heated for 10 minutes. The reaction was cooled, drawn out through the dip tube, the reaction vessel was rinsed with water (1500 μl), added to the glass vial containing the main crude reaction. The whole reaction was loaded on the semi prep HPLC loop and purification started (see below for conditions). The [¹⁸F]F(CH₂)₂0Ts cut peak (retention time 8 minutes) was diluted to a volume of ca.20 ml with water, loaded onto a conditioned light t-C18 sep pak and flushed with H₂O (1×2 ml). The sep pak was dried on a high pressure nitrogen gas line for 20 minutes.

A Wheaton vial containing a stirrer, Precursor Compound 2 (5 mg), Cs₂CO₃ (10 mg) in DMF(100 μl) was stirred at room temperature for 1-2 h. The [¹⁸F]F(CH₂)₂OTs was eluted with CH₃CN (0.5 ml) into the Wheaton vial. The reaction was heated and stirred in an oil bath at 120-130° C. for 15 minutes. After, the reaction was cooled, and quenched with water (500 μl). The whole reaction was loaded onto the HPLC system and the product was purified using the conditions described below (retention time 11 minutes).

The cut peak was diluted with water (10 mL) and was trapped onto a pre-conditioned sep pak t-C18 light using a vacuum pump. The trapped material was washed with water (2 mL) and eluted with ethanol (0.7 mL) and phosphate buffered saline (6.3 mL).

18.4% end of synthesis yield. 2.2 μg of cold ligand total.>99% radiochemical purity.

Prep HPLC system details: HPLC Column HICHROM ACE 5 C18 column, 5u, 100×10 mm; Solvent A=Water, B=MeOH; Flow rate 3 mL/min; UV 254 nm Loop 5 mL.

HPLC Conditions for [¹⁸F]FEtOTs cut: 0-1 mins 50%(B); 1-25 mins 50-95%(B); 25-30 mins 95%; 30-31 mins 95-50%(B); 31-33 mins 50% (B).

HPLC Conditions for [¹⁸F]Compound 2: 0-1 mins 30% (B); 1-20 mins 30-95% (B); 20-25 mins 95%(B); 25-26 mins 95-30% (B); 26-28 mins 30% (B).

Analytical HPLC: HPLC Column Luna C8(2) 150×4.6 mm; Solvent A =Water, B=MeCN; Flow rate 1 mL/min; UV 254 nm; Loop 100 μL.

### Example 5: In Vitro Affinity Assay

To assess affinity of compound of the invention, a competitive radioligand binding assay was carried out that utilised tritiated FMZ as the competitive agent. Tritiated flumazenil was purchased from NEN Perkin Elmer (Cat. NET757250UC) at a concentration of 1 mCi/mL. Briefly, 10 μl of test compound was incubated with a crude homogenate of rat cerebellum in the presence of 2 nM tritiated FMZ (diluted to 40 nM). Homogenate was prepared by homogenisation of cerebellum with Dounce homogenizer in 10× vol homogenization buffer (10 mM KH₂PO₄ buffer pH 7.4). The homogenate was centrifuged at 48,000 g (using SW40Ti rotor=19561 RPM) 30 min at 4° C. The homogenate was kept on ice at all times. After 90 min the assay was filtered through a glass fibre mat, thereby filtering out the rat homogenate and the ligand that has become bound to it. The amount of activity on the filter mat was then measured using liquid scintillation. The affinity data for Compounds 1 and 2, along with the commercially-available prior art compound flumazenil is presented in Table 1 below:

TABLE 1
In vitro affinity data for FMZ (flumazenil) and analogues of FMZ.

| | FMZ | Compound 1 | Compound 2 |
|---|---|---|---|
| Ki (nM) | 0.5 | 2.4 | 0.52 |

### Example 6: In Vivo Biodistribution of [¹⁸F]-Compound 1

Adult male Sprague-Dawley rats (body weight 202±37 g; mean±SD) were injected with between 1 and 5 MBq of [¹⁸F]-Compound 1 via a lateral tail vein. All animals were conscious, but lightly restrained during injection and subsequently housed in short-term metabolism cages. At the appropriate time point; 30 seconds, 2, 10, 30 and 60 minutes post-injection (pi) (n=3 per time point), the animals were sacrificed by cervical dislocation. The brain and peripheral tissues or fluids were sampled post-mortem. Radioactivity in the brain samples was measured using a Wallac gamma counter. Once assayed, the brain samples, along with the remaining organ or tissue samples were assayed using a twin-crystal gamma-counter system (BASIL), with automatic correction for radioactive decay. Table 2 below shows the data obtained in the brain regions.

TABLE 2
Regional brain distribution data following administration of
[¹⁸F]-Compound 1 in naïve male Sprague-Dawley rats.

| | Distribution of [¹⁸F]-Compound 1 | | | | |
|---|---|---|---|---|---|
| | Time Post-Injection [minutes (standard deviation)] | | | | |
| Brain Region (% id/g) | 0.5 | 2 | 10 | 30 | 60 |
| Striatum | 0.26 (0.07) | 0.41 (0.04) | 0.36 (0.04) | 0.21 (0.04) | 0.18 (0.04) |
| Cerebellum | 0.28 (0.06) | 0.43 (0.03) | 0.36 (0.05) | 0.23 (0.04) | 0.20 (0.05) |
| Hippocampus | 0.26 (0.08) | 0.43 (0.06) | 0.43 (0.07) | 0.32 (0.05) | 0.26 (0.07) |
| Pre-frontal cortex | 0.33 (0.1) | 0.57 (0.02) | 0.53 (0.09) | 0.35 (0.07) | 0.24 (0.06) |
| Thalamus | 0.30 (0.11) | 0.47 (0.07) | 0.39 (0.05) | 0.24 (0.05) | 0.20 (0.07) |
| Pituitary gland | 0.60 (0.1) | 0.69 (0.13) | 0.55 (0.10) | 0.30 (0.02) | 0.26 (0.08) |
| Pons/Medulla | 0.23 (0.04) | 0.34 (0.02) | 0.28 (0.05) | 0.18 (0.04) | 0.19 (0.05) |
| Pre-frontal cortex: | 1.13 | 1.23 | 1.35 | 1.44 | 1.21 |
| thalmus | | | | | |

Data expressed as mean (±SD), and all are n = 3.
Data indicated by an asterisk (*) is % id/g.

Whole brain uptake of [¹⁸F]-Compound 1 peaked at 0.9% at 10 minutes pi, with a subsequent clearance that was slow with a decreasing rate (towards a plateau). There was good regional differentiation (between GABA-rich and GABA-poor regions of the brain) that remained apparent at 30 minutes pi.

### Example 7: In Vivo Biodistribution of [¹⁸F]-Compound 2

The biodistribution protocol described in Example 6 for Compound 1 was used to assess Compound 2. Table 3 below shows the data obtained in the brain regions.

TABLE 3
Regional brain distribution data following administration of
[¹⁸F]-Compound 2 in naïve male Sprague-Dawley rats.

| | Distribution of [¹⁸F]-Compound 2 | | | | |
|---|---|---|---|---|---|
| | Time Post-Injection [minutes (standard deviation)] | | | | |
| Brain Region (% id/g) | 0.5 | 2 | 10 | 30 | 60 |
| Striatum | 0.63 (0.05) | 0.71 (0.16) | 0.61 (0.10) | 0.55 (0.096) | 0.45 (0.02) |
| Cerebellum | 0.75 (0.10) | 0.82 (0.19) | 0.74 (0.08) | 0.59 (0.06) | 0.47 (0.02) |
| Hippocampus | 0.63 (0.08) | 0.89 (0.20) | 0.94 (0.06) | 0.75 (0.03) | 0.53 (0.01) |
| Pre-frontal cortex | 0.80 (0.05) | 1.07 (0.29) | 1.04 (0.15) | 0.75 (0.06) | 0.51 (0.03) |
| Thalamus | 0.67 (0.12) | 0.78 (0.20) | 0.69 (0.12) | 0.62 (0.12) | 0.49 (0.02) |
| Pituitary gland | 0.82 (0.21) | 1.07 (0.19) | 0.69 (0.11) | 0.64 (0.04) | 0.51 (0.04) |
| Pons/Medulla | 0.58 (0.09) | 0.61 (0.14) | 0.56 (0.09) | 0.52 (0.05) | 0.45 (0.02) |
| Pre-frontal cortex: | 1.22 | 1.36 | 1.51 | 1.23 | 1.05 |
| thalmus | | | | | |

Data expressed as mean (±SD), and all are n = 3.

Whole brain uptake of [¹⁸F]-Compound 2 peaked at 0.82% at 2 minutes pi, with a subsequent clearance that was slow with a decreasing rate (towards a plateau). There was good regional differentiation (between GABA-rich and GABA-poor regions of the brain) that remained apparent at 10 minutes pi.

### Comparative Example 8:Synthesis of [¹⁸F]-Flumazenil ([¹⁸F]-FMZ)

### Example 8(i): Synthesis of 4-Methyl-7-nitro-3,4-dihydro-1H-benzo[e][1,4]diazepine-2,5-dione (10)

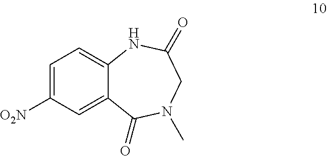

Commercially-available 5-Nitroisatoic anhydride (40 g, 0.192 mol) was dissolved in DMSO (50 mL) by stirring and heating the flask slowly to 140° C. Sarcosine (17.1 g, 0.192 mol) was slowly added in portions to the solution. Upon addition, at 140° C., the solution started bubbling (generation of CO₂). The mixture was left stirring for 2.5 h. The mixture was left to cool and slowly poured on ice cold water in a beaker. The solution was stirred with a glass rod and a yellow solid precipitated out. The solid was separated by filtration and washed several time with water, then dried in vacuum oven at 40° C. overnight. The yellow solid isolated was identified as the desired product 10 in a 78% yield.

¹H NMR (D₆-DMSO): δ 3.14 (3H, s, NCH₃), 3.97 (2H, s, NCH₂CO), 7.30 (1H, d, J=9 Hz, HNCCHCH), 8.33 (1H, dd, J=9 and 3 Hz, CHCHCNO₂CH), 8.33 (1H, d, J=3 Hz, OC—CCH), 11.05 (1H, s, NH).

### Example 8(h): Preparation of Nitromazenil (11)

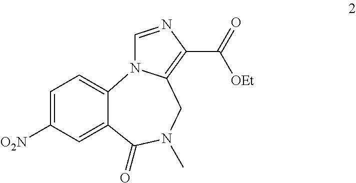

Potassium tert-butoxide (0.6 g, 5 mmol) was added to a solution of intermediate 10 (1 g, 4.3 mmol) in THF (10 mL) and DMF (2 mL) at 0° C. under nitrogen. After 30 min the reaction was cooled to 0° C., treated dropwise with diethyl chlorophosphate (0.7 mL, 5 mmol) and stirred for 30 min. Meanwhile to a stirred solution of ethyl isocyanoacetate (0.6 mL, 5 mmol) in THF (10 mL) under nitrogen at 0° C. was added potassium tert-butoxide (0.6 g, 5 mmol) and stirred for 15 min. This was then added slowly to the mixture of intermediate 10 at 0° C. This was stirred at 0° C. for 0.5 h then at room temperature for another 2 h. TLC (ethyl acetate) showed starting material (Rf 0.4) and a new spot (Rf 0.2) by UV and KMnO₄.

The reaction was quenched with acetic acid and left stirring overnight. The reaction mixture was poured into ice/water. This was extracted with ethyl acetate, and the organic layer was washed with water, brine, dried and concentrated to a thick dark dense oil. This was chromatographed on several times using the following conditions:

1) Companion, using DCM1/ethyl acetate (twice)

2) Companion using petrol/ethyl acetate (twice)

50 mg of the pure material 11 was obtained as a colourless solid (yield 4%)

¹H NMR (CDCl₃): δ 1.39 (3H, t, J=7 Hz, CH₃), 3.28 (3H, s, ArCONCH₃), 4.37 (2H, q, J=7 Hz, OCH₂), 4.40 (1H, br s, CH₂), 5.26 (1H, br s, CH₂), 7.60 (1H, d, J=8.9 Hz, ArCHCHCNO₂), 7.94 (1H, s, NCHN), 8.45 (1H, dd, J=8.9 and 2.8 Hz, ArCHCHCNO₂), 8.95 (1H, d, J=2.5 Hz, ArCHCNO₂).

### Example 9(iii): Radiofluorination of Nitromazenil (11) to Obtain [¹⁸F] flumazenil ([¹⁸ F]FMZ)

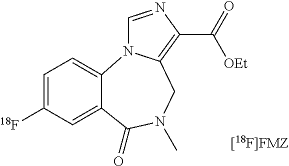

¹⁸F labeling was done on a TRACERlab automated synthesis module (GE Healthcare). [¹⁸F] fluoride was trapped on a pre-conditioned QMA cartridge and then transferred to the reaction vessel using a solution of tetra-n-butylammonium bicarbonate in MeCN/water (MeCN 1400 μL, water 100 μL, TBA.HCO₃ 27 mg) from vial 1. The solution was dried at 100° C. for 10 minutes then 120° C. for 20 minutes using nitrogen plus vacuum flow and then cooled to 50° C.

To the dried [¹⁸F]fluoride was added nitromazenil (18.8 mg) in DMF (1 mL) from vial 3. The reaction mixture was heated at 160° C. for 30 min then it was cooled to 50° C. The reaction mixture was diluted with 10 mM phosphoric acid (2.5 mL) from vial 5 and was transferred to the crude product tube.

The crude product was then transferred onto the preparative HPLC loop manually. Preparative HPLC gave a peak with retention time 17.5 minutes which was cut using into the TRACERlab round bottomed flask containing water (12 mL). The prepartative HPLC system was fitted with a liquid flow scintillation counter.

| HPLC Column | Phenomenex Luna C18(2) 250 × 10 mm 5μ | |
|---|---|---|
| Solvent | A = 10 mM phosphoric acid, | |
| | B = MeCN, 25% B isocratic | |
| Flow rate | 4 | mL/min |
| UV | 254 | nm |
| Loop | 5 | mL |

| | |
|---|---|
| Sensitivity | 2000K |

The mixture in the round bottom flask was trapped on a tC18 plus lite SPE cartridge (pre conditioned with 1 mL ethanol then 2 mL water). The SPE cartridge was washed with water (3 mL) and the crude product eluted into a P6 vial using EtOH (0.5 mL) and water (4.5 mL).

| | | |
|---|---|---|
| Initial activity | 193.8 MBq | @11:14 |
| Activity of formulated product | 14.8 MBq | @12:48 |
| =7.7% end of synthesis yield | | |

What is claimed is:

1) A radiofluorinated compound of Formula I: 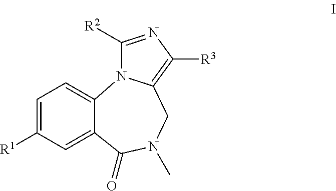
wherein:
one of R¹ or R² is C₁₋₄ [¹⁸F]fluoroalkyl or C₁₋₄ [¹⁸F]fluoroalkoxy, and the other is hydrogen; and,
R³ is C(═O)—O—R⁴ wherein R⁴ is hydrogen, or straight- or branched-chain C₁₋₄ alkyl; or, R⁴ is a C₃₋₅ heterocycle.

2) The radiofluorinated compound as defined in claim 1 wherein one of R¹ and R² is C₁₋₄ [¹⁸F]fluoroalkyl.

3. (canceled)

4. (canceled)

5. (canceled)

6. (canceled)

7. (canceled)

8) The radiofluorinated compound as defined in claim 1 wherein R³ is C(═O)—O—R⁴ wherein R⁴ is straight-chain C₁₋₄ alkyl.

9. (canceled)

10. (canceled)

11) The radiofluorinated compound as defined in claim 1 wherein R³ is C(═O)—O—R⁴ wherein R⁴ is branched-chain C₁₋₄ alkyl.

12. (canceled)

13) A method for the synthesis of a radiofluorinated compound of Formula I as defined in claim 1, wherein said method comprises reaction with a suitable source of ¹⁸F of a precursor compound of Formula Ia: 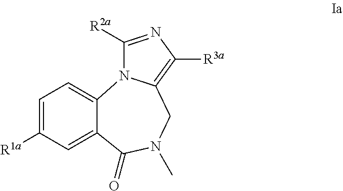
wherein:
one of R^{1a} and R^{2a} is a precursor group, and the other is H, wherein when R^{1a} is a precursor group it is selected from C₁₋₄ alkyl-LG, C₁₋₄ alkoxyl-LG and hydroxyl, and wherein when R^{2a} is a precursor group it is selected from C₁₋₄ alkyl-LG and C₁₋₄ alkoxyl-LG, wherein LG is a leaving group selected from bromide, mesylate or tosylate; and,
R^{3a} is C(═O)—O—R⁴ wherein R⁴ is hydrogen, or straight- or branched-chain C₁₋₄ alkyl; or, R⁴ is a C₃₋₅ heterocycle.

14) The method as defined in claim 13 wherein R^{1a} is said precursor group.

15. (canceled)

16. (canceled)

17) The method as defined in claim 13 wherein said method is automated.

18) A cassette for carrying out the method as defined in claim 17 comprising:
(i) a vessel containing a precursor compound of Formula Ia: 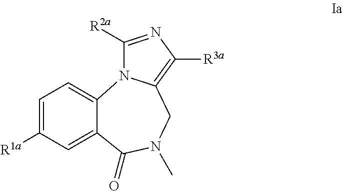
wherein:
one of R^{1a} and R^{2a} is a precursor group, and the other is H, wherein when R^{1a} is a precursor group it is selected from C₁₋₄ alkyl-LG, C₁₋₄ alkoxyl-LG and hydroxyl, and wherein when R^{2a} is a precursor group it is selected from C₁₋₄ alkyl-LG and C₁₋₄ alkoxyl-LG, wherein LG is a leaving group selected from bromide, mesylate or tosylate; and,
R^{3a} is C(═O)—O—R⁴ wherein R⁴ is hydrogen, or straight- or branched-chain C₁₄ alkyl; or, R⁴ is a C₃₋₅ heterocycle; and
(ii) means for eluting the vessel with a suitable source of ¹⁸F.

19. (canceled)

20) A radiopharmaceutical composition comprising the radiofluorinated compound as defined in claim 1 together with a biocompatible carrier in a form suitable for mammalian administration.

21) The radiofluorinated compound as defined in claim 1 for use in a method of PET imaging.

22) A positron emission tomography (PET) imaging method for determining the distribution of GABA_{A} receptors in the central nervous system (CNS) of a subject comprising:
(i) administering to said subject the radiofluorinated compound as defined in claim 1;
(ii) allowing said administered radiofluorinated compound of step (i) to bind to GABA_{A} receptors in the CNS of said subject;
(iii) detecting signals derived from the positron emission decay of the ¹⁸F present in said bound radiofluorinated compound of step (ii); and,
(iv) generating an image of the location and amount of said signals, wherein said signals represent the distribution of GABA_{A} receptors in said subject.

23) The PET method as defined in claim 22 wherein said radiofluorinated compound is administered as a radiopharmaceutical composition comprising said radiofluorinated compound together with a biocompatible carrier in a form suitable for mammalian administration.

24. (canceled)

25) The PET method as defined in claim 22 which is carried out repeatedly during the course of a treatment regimen for said subject, said treatment regimen comprising administration of a drug to combat a GABA_{A} condition.

26) The PET method as defined in claim 22, further comprising step (v) of attributing the distribution of GABA_{A} expression to a particular clinical picture.

27. (canceled)
